(19) United States
(12) Patent Application Publication
(10) Pub. No.: US 2012/0082627 A1
(43) Pub. Date: Apr. 5, 2012

(54) OTIC FOAM FORMULATIONS

(75) Inventors: Rodrigo Yelin, Zur-Yigal (IL); Eran Eilat, Herzliya (IL)

(73) Assignee: OTIC Pharma Ltd., Ariel (IL)

(21) Appl. No.: 13/377,090

(22) PCT Filed: Jun. 8, 2010
(86) PCT No.: PCT/IL2010/000451

§ 371 Date: Dec. 8, 2011

Related U.S. Application Data
(60) Provisional application No. 61/184,853, filed on Jun. 8, 2009.

(51) Int. Cl. A61K 9/12; A61K 31/5383; A61K 31/4709; A61P 31/10; A61K 31/56; A61P 27/16; A61P 31/04; A61K 31/496; A61K 31/5395

(52) U.S. Cl. 424/45; 424/43

(57) ABSTRACT

The present invention relates to foamable otic pharmaceutical compositions comprising fluoroquinolones and methods of preparing same. Particularly, the present invention relates to pharmaceutical compositions comprising oil-in-water emulsions comprising fluoroquinolones and gas propellants, administered to the ear as foam for treating ear disorders.

### FIELD OF THE INVENTION

The present invention relates to foamable otic pharmaceutical compositions comprising fluoroquinolones and methods of preparing same. Particularly, the present invention relates to pharmaceutical compositions comprising oil-in-water emulsions comprising fluoroquinolones and gas propellants, administered to the ear as foam for treating ear disorders.

### BACKGROUND OF THE INVENTION

Otitis externa which involves the ear canal portion of the external ear is a common otologic problem occurring mainly during hot and humid weather. Otitis externa is five times more frequent in swimmers than in non-swimmers. It is an acute or chronic inflammation of the epithelium of the external ear canal. It may develop anywhere from the tympanic membrane to the pinna. It is variably characterized by erythema, edema, increased sebum or exudates, and desquamation of the epithelium. In later stages, suppuration occurs in the ear canal and hearing may be decreased. Over 90% of cases of acute Otitis Externa (AOE) are due to bacterial and fungal infections.

Otitis media involves infections of the middle ear and it is a very common otologic problem in adults and particularly in children. It has been estimated that nearly 95% of all children experience one or more episodes of otitis by age 9, and that about 15% of all visits by children to pediatricians are in regard to otitis media. In children, the disease is often associated with upper respiratory afflictions which trigger a transudate secretion response in the Eustachian tube and middle ear. Bacteria and viruses migrate from the naso-pharynx to the middle ear via the Eustachian tube, and can cause the Eustachian tube to become blocked, preventing ventilation and drainage of the middle ear.

Otitis externa is the most common disease of the ear canal in dogs and cats, and is occasionally seen in rabbits (in which it is usually due to the mite Psoroptescuniculi).

The common treatment of AOE consists of topical antibiotics, with or without steroids, analgesia and water avoidance. Otic preparations are generally supplied in the form of ear drops. In advanced cases, when suppuration blocks the external ear canal, a wick is inserted and soaked in the topical preparation. However, its insertion may be traumatic, inconvenient and painful. Antibiotic agents in ear drops include aminoglycosides (mainly neomycin) in combination with polymyxin B and hydrocortisone or fluoroquinolones such as ciprofloxacin and ofloxacin. The incidence of hypersensitivity reactions to neomycin-containing ear drops has been reported to be as high as 13%, while such reactions to fluoroquinolones have been reported to be rare. In addition, aminoglycosides have the potential to damage the inner ear when the tympanic membrane is perforated, while ototoxicity is not a concern with fluoroquinolones.

Ciprofloxacin is a safe and efficacious antibacterial fluoroquinolone active against a broad spectrum of gram-positive and gram-negative bacteria. Ciprofloxacin is present as Ciprofloxacin base and Ciprofloxacin HCl. Ciprofloxacin HCl is the monohydrochloride monohydrate salt of 1-cyclopropyl-6-fluoro-1,4-dihydro-4-oxo-7-(1-piperazinyl)-3-quinolinecarboxylic acid with the following chemical structure:

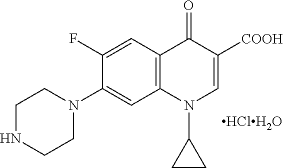

Due to its proven safety and lack of ototoxicity, ciprofloxacin ear drops are prescribed to treat patients with Acute Otitis Externa with intact or non-intact tympanic membrane. For example, topical otic compositions containing a combination of either ciprofloxacin and hydrocortisone or ciprofloxacin and dexamethasone are sold under the name of CIPRO HC™ and CIPRODEX™, respectively, by Alcon Laboratories, Inc.

U.S. Pat. No. 5,843,930 to Purwar et al., discloses a non-ototoxic, topical, otic pharmaceutical composition for the treatment of otitis externa and otitis media which comprises ciprofloxacin, a viscosity augmenter, and water sufficient to produce a liquid composition. According to U.S. Pat. No. 5,843,930, the composition can be a suspension comprising ciprofloxacin, hydrocortisone, polyvinyl alcohol as a viscosity augmenter, lecithin to enhance suspension of other constituents, benzyl alcohol as a preservative, sodium acetate and acetic acid as a buffering system, polysorbate 20 to 80 to spread the composition on a hydrophobic skin surface, and water sufficient to produce a liquid composition. U.S. Pat. No. 5,843,930 further discloses methods for treating otitis which comprise administering the otic pharmaceutical composition topically into the ear.

U.S. Pat. No. 6,284,804 to Singh et al., discloses suspension formulations comprising ciprofloxacin, dexamethasone, sodium chloride as an ionic tonicity agent, a nonionic polymer, and a nonionic surfactant. The formulations according to U.S. Pat. No. 6,284,804 have a pH from 3-5 that can be adjusted by NaOH/HCl and comprise the buffering system of sodium acetate and acetic acid. According to U.S. Pat. No. 6,284,804, the formulations may further comprise quaternary ammonium halide as a preservative and a chelating agent.

U.S. Pat. No. 6,462,033 to Singh discloses a composition comprising ciprofloxacin, hydrocortisone, a preservative which must be jointly soluble with ciprofloxacin in water, a tonicity adjusting agent to adjust the tonicity of the composition to 200-600 mOsm, and lecithin. According to U.S. Pat. No. 6,462,033, a buffering system, preferably acetate buffer, a nonionic surfactant and polyvinyl alcohol are desirable. The compositions of U.S. Pat. No. 6,462,033 have excellent physical stability attributed to the method of their preparation, i.e., hydrocortisone is dispersed with lecithin and optionally with a polysorbate surfactant for greater than 45 minutes prior to combining hydrocortisone with the remainder of the composition.

U.S. Patent Application Publication No. 2006/0269485 to Friedman et al., discloses a foamable composition which includes: an antibiotic agent; at least one organic carrier selected from the group consisting of a hydrophobic organic carrier, an organic polar solvent, an emollient, and mixtures thereof, at a concentration of about 2% to 50% by weight; a surface active agent at a concentration of about 0. 1% to about 5% by weight; at least one polymeric additive selected from the group consisting of a bioadhesive agent, a gelling agent, a film forming agent, and a phase change agent; water; and liquefied or compressed gas propellant at a concentration of about 3% to about 25% by weight of the total composition. The antibiotic agent according to U.S. Patent Application Publication No. 2006/029485 is selected from the group consisting of beta lactam antibiotics, aminoglycosides, ansa-type antibiotics, anthraquinones, antibiotic azoles, antibiotic glycopeptides, macrolides, antibiotic nucleosides, antibiotic peptides, antibiotic polyenes, antibiotic polyethers, quinolones, antibiotic steroids, sulfonamides, tetracycline, dicarboxylic acids, antibiotic metals, oxidizing agents, and others. According to U.S. Patent Application Publication No. 2006/029485, the composition can be an oil-in-water emulsion or water-in-oil emulsion.

International Application Publication No. WO 2005/055921 to one of the inventors of the present application discloses a pharmaceutical composition for the treatment of an ear disorder in a form selected from foam and mousse comprising: a pharmaceutical agent known to affect an ear disorder; a pharmaceutically acceptable carrier comprising a dispersing agent that is a foam forming agent; a dispensing device adapted for the dispensing of the pharmaceutical agent admixed with the carrier to the external auditory meatus.

There is an unmet need for foamable otic, clinically efficacious, and stable compositions comprising fluoroquinolones which can be readily and compliantly used by a patient for the treatment an ear disorder.

### SUMMARY OF THE INVENTION

The present invention provides foamable compositions comprising fluoroquinolones useful for otic administration. Particularly, the present invention provides otic foamable compositions comprising as an active agent ciprofloxacin for treating otitis. The present invention further provides methods for preparing same and use thereof.

The present invention is based in part on the surprising finding that ciprofloxacin formulated in foam is particularly stable and useful for topical applications into the ear.

It is now disclosed for the first time that foam formulations comprising ciprofloxacin were preferable over commercially available ear drops of ciprofloxacin as the foam formulations did not cause dizziness or reduce the hearing capability in human subjects treated with these formulations. The foam formulations comprising ciprofloxacin did not cause a cold sensation as commercially available ear drops of ciprofloxacin but rather a comfortable and warm sensation when applied into the ear of a human subject. The foam formulations comprising ciprofloxacin did not cause skin irritation or any allergic response and as such they were safe for use. The antibacterial efficacy of ciprofloxacin in the foam formulations was identical to that obtained by the commercially available ear drops of ciprofloxacin.

Moreover, the foam formulations comprising ciprofloxacin collapsed slowly in the ear forming a tube-like structure of which the center of the foam became hollow while the tube walls (adjacent to the ear canal walls) remained stable for long periods of time. Such foam formulations provided a slow-release effect leading to an extended exposure of the ear canal to ciprofloxacin as compared to the effect obtained by commercially available ear drops of ciprofloxacin.

The foam formulation did not spill out of the ear as typically happens with ear drops and as such otic foam formulations comprising ciprofloxacin delivered accurate dosage of the antibiotic agent. The foamable formulations comprising ciprofloxacin were highly stable after long term storage showing no significant effect on ciprofloxacin content, foam quality, foam density or foam collapse rate. Thus, the foamable formulations comprising ciprofloxacin of the present invention are highly efficacious for otic application.

The compositions of the present invention are oil-in-water emulsions. As such, the compositions comprise water-soluble or poorly water-soluble fluoroquinolones and can further comprise hydrophobic compounds, e.g., steroids such as dexamethasone or hydrocortisone useful for alleviating or treating inflammatory reactions. It is to be appreciated that the foamable compositions of the present invention comprise at least 50% water.

It is now disclosed that the amount of a compressed propellant gas in the otic foamable compositions can be designed so as to form foam having a density of at least 0.1 gr/ml to about 0.5 gr/ml which brings about to foam collapse within about 30 minutes to about 6 hours after administration of the foam into the ear.

According to a first aspect, the present invention provides a foamable otic pharmaceutical composition comprising:
(a) an oil-in-water emulsion comprising:
   (i) a fluoroquinolone or a salt thereof in an amount effective for antibacterial action;
   (ii) a hydrophobic solvent;
   (iii) an emulsifier and/or a synthetic surfactant;
   (iv) a stabilizing agent;
   (v) a polar co-solvent in an amount ranging from about of 5% to about 30% (w/w);
   (vi) water in an amount ranging from about 50% to about 80% (w/w); and
(b) a compressed propellant gas;

wherein the composition packaged in a container is adapted to form foam after dispensing from the container, the foam having a density of about 0.1 gr/ml to about 0.5 gr/ml. Preferably, the foamable otic pharmaceutical composition further comprises a buffering system, and optionally a preservative.

It is to be understood that the foamable otic pharmaceutical composition of the present invention is stored in an aerosol or pressurized container. Upon dispensing from the aerosol container, the foamable otic pharmaceutical composition forms foam suitable for application into the ear. Thus, the pharmaceutical composition of the present invention when administered to the ear of a subject, e.g., a human or an animal, is in the form of foam.

It is to be further understood that the amount of the compressed propellant or liquefied gas is adapted to provide foam collapse within about 30 minutes to about 6 hours after administration of the foam into the ear of a subject. Alternatively, the amount of the compressed propellant or liquefied gas is adapted to provide foam collapse within about 5 hours, and further alternatively within about 3 hours after administration of the foam into the ear of a subject. Accordingly, the amount of the compressed propellant gas is adapted to provide foam density of about 0.1 gr/ml to about 0.3 gr/ml, alternatively from about 0.11 gr/ml to about 0.2 gr/ml, and further alternatively from about 0.12 gr/ml to about 0.17 gr/ml.

According to some embodiments, the pharmaceutical composition comprises a fluoroquinolone selected from the group consisting of ciprofloxacin, ofloxacin, moxifloxacin, levofloxacin, lomefloxacin, nadifloxacin, norfloxacin, pefloxacin, rufloxacin, balofloxacin, gatifloxacin, grepafloxacin, levofloxacin, pazufloxacin, sparfloxacin, temafloxacin, tosufloxacin, clinafloxacin, gemifloxacin, sitafloxacin, trovafloxacin, prulifloxacin, garenoxacin, delafloxacin, marbofloxacin, enrofloxacin, danofloxacin, difloxacin, ibafloxacin, orbifloxacin, sarafloxacin and salts thereof. According to further embodiments, the fluoroquinolone is selected from the group consisting of ciprofloxacin, ofloxacin, moxifloxacin, levofloxacin, marbofloxacin, enrofloxacin, and salts thereof. According to a certain embodiment, the fluoroquinolone is ciprofloxacin or a salt thereof. According to a further embodiment, the composition is for animal use and the fluoroquinolone is marbofloxacin or enrofloxacin.

According to some embodiments, the oil-in-water emulsion comprises: (i) ciprofloxacin in an amount ranging from about 0.1% to about 0. 5% (w/w); (ii) a hydrophobic solvent; (iii) an emulsifier; (iv) a synthetic surfactant; (v) a stabilizing agent; (vi) a polar co-solvent in an amount ranging from about 5% to about 30% (w/w); (vii) a buffering system; and (viii) water in an amount ranging from about 50% to about 80% (w/w).

According to additional embodiments, the oil-in-water emulsion comprises: (i) ciprofloxacin in an amount ranging from about 0.2% to about 0.35% (w/w); (ii) a hydrophobic solvent in an amount ranging from about 5% to about 15% (w/w); (iii) an emulsifier in an amount ranging from about 0.1% to about 10% (w/w); (iv) a synthetic surfactant in an amount ranging from about 0.1% to about 10% (w/w); (v) a stabilizing agent in an amount ranging from about 0.1% to about 10% (w/w); (vi) a polar co-solvent in an amount ranging from about 5% to about 30% (w/w); (vii) a buffering system; and (viii) water in an amount ranging from about 50% to about 80% (w/w). The foamable otic pharmaceutical composition optionally comprises a preservative.

According to an exemplary embodiment, ciprofloxacin is ciprofloxacin hydrochloride present in the pharmaceutical composition in an amount of about 0.35% (w/w). According to another embodiment, ciprofloxacin is ciprofloxacin base.

According to a further embodiment, the oil is selected from the group consisting of mineral oil and medium chain triglyceride (MCT) oil. According to some embodiments, the amount of the mineral oil in the pharmaceutical composition ranges from about 5% to about 15% (w/w), alternatively from about 5% to about 10%. According to a certain embodiment, the amount of the mineral oil is of about 6% (w/w). According to another embodiment, the hydrophobic solvent is isopropyl myristate, preferably present in the composition in an amount of about 10% (w/w).

According to additional embodiments, the emulsifier is selected from the group consisting of lecithin or a derivative thereof, phospholipids such as phosphatidylcholine and phosphatidylethanolamine, long chain alcohols having at least 12 carbon atoms in the carbon chain such as cetyl alcohol and cetostearyl alcohol, and combinations thereof. According to an exemplary embodiment, the amount of the emulsifier in the pharmaceutical composition ranges from about 1% to about 5% (w/w).

According to further embodiments, the synthetic surfactant is selected from the group consisting of glyceryl stearate, polysorbate 20, polysorbate 60, polysorbate 80, polyoxyl 40 stearate, and combinations thereof. According to a certain embodiment, the amount of the surfactant in the pharmaceutical composition ranges from about 3% to about 4% (w/w).

According to yet further embodiments, the stabilizing agent is selected from the group consisting of hydroxyethyl cellulose, polyvinyl alcohol, and a combination thereof According to a certain embodiment, the amount of the stabilizing agent in the pharmaceutical composition ranges from about 1% to about 2% (w/w).

According to still further embodiments, the polar co-solvent is selected from the group consisting of propylene glycol, glycerin, polypropylene glycol stearyl ether, and combinations thereof. According to a certain embodiment, the amount of the polar co-solvent in the pharmaceutical composition ranges from about 6% to about 16% (w/w).

According to yet further embodiments, the buffering system is selected from the group consisting of acetate buffer, citrate buffer, and phosphate buffer. According to a certain embodiment, the buffering system comprises sodium acetate and acetic acid. According to an exemplary embodiment, the amount of sodium acetate in the pharmaceutical composition ranges from about 0.1% to about 1% (w/w), preferably the amount is of about 0.2% (w/w).

According to another embodiment, the amount of water in the pharmaceutical composition ranges from about 60% to about 80%, alternatively from about 70% to about 80% (w/w).

According to still further embodiments, the pH of the pharmaceutical composition ranges from about 4 to about 7. Alternatively, the pH of the composition ranges from about 4 to about 5. According to a certain embodiment, the pH of the pharmaceutical composition ranges from about 4.5 to about 5.

According to yet further embodiments, the propellant gas is volatile hydrocarbons such as butane, propane, isobutane, and mixture thereof The amount of the compressed propellant gas in the pharmaceutical composition ranges from about 1% to about 8% by weight of the composition, alternatively from about 2% to about 6%, and further alternatively from about 4% to about 6% by weight of the composition. It is to be understood that gas propellants such as, for example, hydrofluoroalkanes, chlorofluoroalkanes, dimethyl ethers, and methyl ethers can be used as propellant gas in the compositions of the present invention.

According to still further embodiments, the foamable otic pharmaceutical composition further comprises a steroid having an anti-inflammatory activity. Among the anti-inflammatory steroids, hydrocortisone, hydrocortisone acetate, dexamethasone, dexamethasone sodium phosphate, prednisolone, methylprednisolone, prednisone, triamcinolone acetonide, mometasone, budesonide, fluocinolone acetonide, betamethasone, betamethasone sodium phosphate, betamethasone valerate, cortisone acetate, isoflupredone acetate, tixocortol pivalate, triamcinolone alcohol, amcinonide, desonide, fluocinonide, halcinonide, fluocortolone, hydrocortisone-17-butyrate, hydrocortisone-17-valerate, aclometasone dipropionate, betamethasone dipropionate, prednicarbate, clobetasone-17-butyrate, clobetasol-17-propionate, fluocortolone caproate, fluocortolone pivalate, fluprednidene acetate can be used. According to certain embodiments, hydrocortisone, hydrocortisone acetate, dexamethasone, and dexamethasone sodium phosphate are preferred. The anti-inflammatory steroid is present in the composition in an amount effective for anti-inflammatory action. Such amount typically ranges from about 0.1% to about 3% (w/w).

According to yet further embodiments, the foamable otic pharmaceutical composition further comprises an analgesic agent. Among the analgesic agents, benzocaine, tetracaine, procaine and antipyrine are preferred.

According to still further embodiments, the composition is for animal use and further comprises an antifungal agent. Among the antifungal agents, nystatin, clotrimazole, miconazole, ketoconazole, fluconazole, thiabendazole, econazole, clomidazole, isoconazole, tiabendazole, tioconazole, sulconazole, bifonazole, oxiconazole, fenticonazole, omoconazole, sertaconazole, flutrimazole can be used. According to certain embodiments, nystatin, clotrimazole, miconazole, ketoconazole, fluconazole, thiabendazole are preferred.

According to yet further embodiments, the foamable otic composition is for animal use and can further comprise an insecticide agent. Among the insecticide agents, pyrethrins, pyrethroids, piperonyl butoxide, and N-octyl bicycloheptene dicarboximide can be used.

According to an exemplary embodiment, the foamable otic pharmaceutical composition comprises: ciprofloxacin HCl in an amount of 0.35% (w/w), mineral oil in an amount of 6% (w/w), cetyl alcohol in an amount of 1% (w/w), lecithin in an amount of 2% (w/w), polysorbate 80 in an amount of 3% (w/w), hydroxyethyl cellulose in an amount of 0.25% (w/w), polyvinyl alcohol in an amount of 1% (w/w), propylene glycol in an amount of 8% (w/w), glycerin in an amount of 8% (w/w), sodium acetate in an amount of 0.2% (w/w), acetic acid, water in an amount of about 70% (w/w), the pH of the pharmaceutical composition is of about 4.5 to about 5 before adding the compressed propellant gas in an amount of 4% to 6% by weight of the composition.

According to another exemplary embodiment, the foamable otic pharmaceutical composition comprises: ciprofloxacin HCl in an amount of 0.35% (w/w), mineral oil in an amount of 6% (w/w), cetyl alcohol in an amount of 1% (w/w), glyceryl stearate in an amount of 0.5% (w/w), lecithin in an amount of 2% (w/w), polysorbate 80 in an amount of 3% (w/w), hydroxyethyl cellulose in an amount of 0.3%, polyvinyl alcohol in an amount of 1.5% (w/w), propylene glycol in an amount of 6% (w/w), glycerin in an amount of 6% (w/w), sodium acetate in an amount of 0.2% (w/w), acetic acid, water in an amount of 74% (w/w), the pH of the pharmaceutical composition is of about 4.5 to about 5 before adding the compressed propellant gas in an amount of 2% to about 4% by weight of the composition.

According to a further exemplary embodiment, the foamable otic pharmaceutical composition comprises: ciprofloxacin HCl in an amount of 0.35% (w/w), mineral oil in an amount. of 10% (w/w), cetyl alcohol in an amount of 1% (w/w), glyceryl stearate in an amount of 0.5% (w/w), polyoxyl 40 stearate in an amount of 2.6% (w/w), polysorbate 80 in an amount of 0.8% (w/w), hydroxyethyl cellulose in an amount of 0.5% (w/w), propylene glycol in an amount of 3 (w/w), glycerin in an amount of 3% (w/w), sodium acetate in an amount of 0.2% (w/w), acetic acid, water in an amount of 78% (w/w), the pH of the pharmaceutical composition is of about 4.5 to about 5 before adding the compressed propellant gas in an amount of 4% to about 6% by weight of the composition.

According to another aspect, the present invention provides a method for preparing a foamable otic pharmaceutical composition comprising admixing a fluoroquinolone, preferably formulated in a solid form, with an oil-in-water emulsion, and admixing a compressed propellant gas with the oil-in-water emulsion in a container, the propellant gas and the oil-in-water emulsion adapted to form foam after dispensing from the container, the foam has a density of about 0.1 gr/ml to about 0.5 gr/ml. Examples of a solid form of fluoroquinolone include powder and crystals. Preferably, the fluoroquinolone is formulated in a powder.

According to some embodiments, the method comprises the following steps:
(a) preparing an aqueous solution comprising water, a stabilizing agent, and optionally a polar co-solvent;
(b) preparing an oil phase solution comprising a hydrophobic solvent, an emulsifier, and optionally a polar co-solvent and/or a synthetic surfactant;
(c) combining the oil phase solution of step (b) with the aqueous solution of step (a) to obtain an emulsion;
(d) admixing a fluoroquinolone with the emulsion of step (c); and
(e) admixing a compressed propellant gas with the emulsion, the amount of the propellant gas is adapted to form foam with the oil-in-water emulsion, wherein the foam having a density of about 0.1 g/ml to about 0.5 g/ml.

According to further embodiments, the method comprises the following steps:
(a) dissolving a stabilizing agent in water to obtain even dispersion of the stabilizing agent;
(b) dissolving a polar co-solvent in the dispersion of step (a) so as to obtain an aqueous solution;
(c) mixing an oil, an emulsifier, and optionally a synthetic surfactant to obtain a clear solution;
(d) dissolving a surfactant in the solution of step (c);
(e) combining the oil phase solution of step (d) with the aqueous solution of step (b) to obtain an emulsion;
(f) adjusting the pH of the emulsion of step (e) to about 4.5 to about 5 with a buffering system;
(g) admixing a fluoroquinolone with the emulsion of step (f);
(h) adjusting the weight of the emulsion of step (g) to 100% with water; and
(i) admixing a compressed propellant gas with the emulsion of step (h).

According to a certain embodiment, the method comprises the following steps:
(a) dissolving stabilizing agents polyvinyl alcohol and hydroxyethylcellulose in water to obtain a dispersion of the stabilizing agents ;
(b) dissolving propylene glycol and glycerin in the dispersion of step (a) so as to obtain a clear aqueous solution;
(c) mixing mineral oil, cetyl alcohol, and glyceryl monostearate to obtain a clear solution;
(d) dissolving polysorbate 80 in the solution of step (c);
(e) dissolving lecithin in the solution of step (d) so as to obtain a clear oil phase solution;
(f) combining the oil phase solution of step (e) with the aqueous solution of step (b) to obtain an emulsion;
(g) adjusting the pH of the emulsion of step (f) to about 4.5 to about 5 with sodium acetate and acetic acid;
(h) admixing ciprofloxacin HCl monohydrate with the emulsion of step (g);
(i) adjusting the weight of the emulsion of step (h) to 100% with water; and
(j) admixing hydrocarbon gas with the emulsion of step (j).

It is to be understood that the emulsion of the invention is typically placed in an aerosol container to which the compressed gas propellant is added so as to obtain the foamable otic pharmaceutical composition.

According to another aspect, the present invention provides a foamable otic pharmaceutical composition prepared by the methods of the present invention.

According to a further aspect, the present invention provides a method for treating an ear disorder comprising administering to the ear of a subject in need of such treatment a therapeutically effective amount of the foamable otic pharmaceutical composition according to the principles of the present invention. According to some embodiments, the subject is a human or an animal. According to further embodiments, the animal is a pet animal. According to certain embodiments, the pet animal is a dog or a cat. According to additional embodiments, the ear disorder is otitis selected from the group consisting of otitis externa (swimmer's ear) and otitis media. According to additional embodiments, the otitis externa is selected from the group consisting of acute otitis externa and suppurative otitis externa. According to further embodiments, the otitis media is selected from the group consisting of chronic suppurative otitis media and serous or secretory otitis media due to tympanostomy tubes.

According to yet further aspect, the present invention provides use of (a) an oil-in-water emulsion comprising: (i) a fluoroquinolone or a salt thereof in an amount effective for antibacterial action; (ii) a hydrophobic solvent; (iii) an emulsifier and/or a synthetic surfactant; (iv) a stabilizing agent; (v) a polar co-solvent in an amount ranging from about of 5% to about 30% (w/w); (vi) water in an amount ranging from about 50% to about 80% (w/w); and (b) a compressed propellant gas; wherein the oil-in-water emulsion and the compressed propellant gas contained in a container are adapted to form foam having a density of about 0.1 gr/ml to about 0.5 gr/ml; for the manufacture of a medicament for treating an ear disorder according to the principles of the present invention.

These and other embodiments of the present invention will be better understood in relation to the figures, description, examples and claims that follow.

### BRIEF DESCRIPTION OF THE FIGURES

FIG. 1 is a photograph showing the in vitro antibiotic efficacy of the foam otic formulations. The inhibition of Escherichia coli growth by filters soaked with Ciloxan® ear drops or with the different foam formulations was evaluated by measuring the diameter of growth inhibition.

### DETAILED DESCRIPTION OF THE INVENTION

The present invention provides a foamable otic pharmaceutical composition comprising: (a) an oil-in-water emulsion comprising: (i) a fluoroquinolone in an amount effective for antibacterial action; (ii) a hydrophobic solvent; (iii) an emulsifier and/or a synthetic surfactant; (iv) a stabilizing agent; (v) a polar co-solvent in an amount ranging from about of 5% to about 30% (w/w); (vi) water in an amount ranging from about 50% to about 80% (w/w); and (b) a compressed propellant gas; the oil-in-water emulsion and the compressed propellant gas disposed in a container are adapted to form foam when dispense from the container, the foam having a density of about 0.1 gr/ml to about 0.5 gr/ml.

The present invention provides foamable otic pharmaceutical compositions for use in treating ear disorders in humans or in animals. It is to be understood that the compositions for animal use can further comprise anti-fungal agents and/or miticide agents.

The term “foam collapse” denotes the kinetics of foam coarsening and destruction resulting from the rupture of bubbles or from gas transfer from small to large bubbles and bubble coalescence occurring within the ear of a subject.

The term “about” as used herein denotes ±10% of the value indicated.

The term a “water-soluble” fluoroquinolone refers to a fluoroquinolone having solubility in water in the range of 1 gr/ml to 1 gr/50 ml at room temperature. The term “poorly water-soluble” fluoroquinolone as used herein refers to a fluoroquinolone that typically has solubility in water in the range of 1 gr/50 ml to 1 gr/10,000 ml at room temperature.

The term “therapeutically effective amount” is that amount of the fluoroquinolone which is sufficient to provide a beneficial effect to the subject to which the fluoroquinolone is administered. More specifically, a therapeutically effective amount means an amount of the fluoroquinolone effective to alleviate or ameliorate the symptoms of an ear disorder of the subject being treated. Among the symptoms of an ear disorder, ear edema, ear pain, ear discharge, and tenderness to movement of the tragus/pinna are more typical.

According to a certain embodiment, the foamable otic pharmaceutical composition comprises ciprofloxacin as the anti-bacterial agent.

According to an exemplary embodiment, the present invention provides a foamable otic pharmaceutical composition of ciprofloxacin comprising: (i) ciprofloxacin in an amount ranging from about 0.3% to about 0.35% (w/w); (ii) a hydrophobic solvent in an amount ranging from about 5% to about 15% (w/w); (iii) an emulsifier in an amount ranging from about 0.1% to about 10% (w/w); (iv) a synthetic surfactant in an amount ranging from about 0.1% to about 10% (w/w); (v) a stabilizing agent in an amount ranging from about 0.1% to about 10% (w/w); (vi) a polar co-solvent in an amount ranging from about of 5% to about 30% (w/w); (vii) a buffering system; (viii) water in an amount ranging from about 50% to about 80% (w/w); and (ix) a compressed propellant gas. The foamable otic pharmaceutical composition optionally comprises a preservative.

It is to be appreciated that the foam formulation comprising ciprofloxacin comprise at least 50% water of the total weight of the composition (w/w), alternatively at least 60% water, further alternatively at least 70% water, and still alternatively at least 75% water of the total weight of the composition.

The compositions of the present invention comprise a hydrophobic solvent. A “hydrophobic solvent” as used herein refers to a material having solubility in distilled water at ambient temperature of less than 1 gr per 100 ml. According to the invention, the hydrophobic solvent can comprise therapeutically active oil. The therapeutically active oil is liquid oil that may originate from vegetable, marine or animal sources. Alternatively, the therapeutically active oil can be a mineral oil. Mineral oil (e.g., Chemical Abstracts Service Registry number 8012-95-1) is a mixture of aliphatic, naphthalenic, and aromatic liquid hydrocarbons that derive from petroleum. Other oils can be polyunsaturated oils containing poly-unsaturated fatty acids (see, for example, US 2006/0269485, the content of which is incorporated by reference as if fully set forth herein).

Alternatively, the hydrophobic solvent include, but are not limited to, isopropyl myristate, isopropyl palmitate, isopropyl isostearate, diisopropyl adipate, diisopropyl dimerate, isopropyl lanolate, myristyl myristate, and triisocetyl citrate.

The composition comprises a polar co-solvent. A “polar co-solvent” is an organic solvent, typically soluble in both water and oil. Examples of polar solvents include, but are not limited to, polyols, such as glycerol (glycerin), propylene glycol (PPG), hexylene glycol, diethylene glycol, PPG n-alkanols, and (PPG) stearyl ether. According to certain embodiments, the polar solvent is a polyethylene glycol (PEG), PPG, or a derivative thereof that is liquid at ambient temperature, including, but not limited to, PEG200 (molecular weight (MW) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

The stabilizing agent is a polymeric agent that increases the viscosity of the composition, contributes to the composition stability, and/or slows the foam collapse rate. Examples of stabilizing agents include, but are not limited to, naturally-occurring polymeric materials (e.g., alginate, albumin, gelatin, carrageenan, xanthan gum, starch), semi-synthetic polymeric materials such as cellulose ethers (e.g. hydroxyethyl cellulose, methyl cellulose, carboxymethyl cellulose, hydroxy propylmethyl cellulose), and synthetic polymeric materials(e.g., polyvinyl alcohol, carboxyvinyl polymers, and polyvinylpyrrolidone).

A surfactant or a surface-active agent include any agent linking oil and water in the composition, in the form of emulsion. A hydrophilic/lipophilic balance (HLB) of a surfactant indicates its affinity toward water or oil. The HLB scale ranges from 1 (totally lipophilic) to 20 (totally hydrophilic), with 10 representing an equal balance of both characteristics. Hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average).

According to one or more embodiments of the present invention, the surface-active agent has a hydrophilic lipophilic balance (HLB) between about 9 and about 14, which is the required HLB (the HLB required to stabilize an O/W emulsion of a given oil) of most oils and hydrophobic solvents. Thus, in one or more embodiments, the composition contains a single surface active agent having an HLB value between about 9 and 14, and in one or more embodiments, the composition contains more than one surface active agents and the weighted average of their HLB values is between about 9 and about 14. Non-limiting examples of possible surfactants are synthetic non-ionic surfactants that include polysorbates, such as polyoxyethylene sorbitan monolaurate (Tween 20), polyoxyethylene sorbitan monostearate (Tween 60) and polyoxyethylene sorbitan monooleate (Tween 80); glyceryl stearate; polyoxyethylene (POE) fatty acid esters, such as Myrj 45, Myrj 49, Myrj 52 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, and the like, and a combination thereof.

The composition of the present invention further comprises an emulsifier as known in the art. It is to be understood that the emulsifier in the compositions of the present invention enables obtaining a clear hydrophobic (oil) solution. The emulsifier is selected from the group consisting of lecithin or a derivative thereof, phospholipids, and fatty alcohols having 12 or more carbons in their carbon chain, such as lauryl alcohol, myristyl alcohol, cetyl alcohol, stearyl alcohol, oleyl alcohol, and linoleyl alcohol, or mixtures thereof Other examples of fatty alcohols are arachidyl alcohol (C20), behenyl alcohol (C22), 1-triacontanol (C30), as well as alcohols with longer carbon chains (up to C50). The composition may further comprise a thickener.

The compositions of the present invention can be subjected to nano-sizing. Methods for nano-sizing of compositions are well known in the art and include, but not limited to, nano-sizing by a high pressure homogenizer.

The composition of the present invention can further comprise a variety of formulation excipients. Such excipients can be selected, for example, from preservatives (e.g., benzyl alcohol), buffering agents (acetate buffer, citrate buffer, phosphate buffer, and the like) antioxidants, humectants, colorant and odorant agents and other formulation components used in the art of formulation.

Gas propellants are used to generate and administer the foamable composition as foam. Examples of suitable gas propellants include volatile hydrocarbons such as butane, propane, isobutane or mixtures thereof, and fluorocarbon gases. The amount of the compressed propellant or liquefied gas is adapted to provide foam collapse within about 30 minutes to about 10 hours after administration of the foam into the ear of a subject. Alternatively, the amount of the compressed propellant or liquefied gas is adapted to provide foam collapse within about 6 hours, further alternatively within about 5 hours, 4 hours or 3 hours after administration of the foam into the ear of a subject. The amount of the compressed propellant gas is adapted to provide foam density of about 0.1 gr/ml to about 0.3 gr/ml, alternatively from about 0.11 gr/ml to about 0.2 gr/ml, and further alternatively from about 0.12 gr/ml to about 0.17 gr/ml.

### EXAMPLE 1

### Solubility of Cyprofloxacin in Different Solvents

The solubility of Ciprofloxacin HCl in solvents suitable for formulation development was measured. The results of the study are summarized in the Table 1.

TABLE 1
Solubility test for Ciprofloxacin HCl.

| Solvent | Ciprofloxacin HCl | |
|---|---|---|
| IPM | Not soluble | |
| Mineral oil | Not soluble | |
| MCT oil | Not soluble | |
| Propylene glycol | <2.5 | mg/ml^{a} |
| Glycerin | Not soluble | |
| Water | 27 | mg/ml^{b} |
| PEG 400 | <2.5 | mg/ml^{a} |
| Buffer Acetate, pH 4.5 | 32.6 | mg/ml^{b} |

^{a}Ciprofloxacin HCl was not soluble above the tested concentration
^{b}Maximal solubility observed

As indicated in Table 1, aqueous buffer acetate was found to be more suitable to dissolve Ciprofloxacin HCl and hence was chosen for further formulation development as a main solvent.

### EXAMPLE 2

### Foam Formulations and Characterization

The different foam formulations were evaluated for foam quality, agitability, pH, appearance after centrifugation, foam density, foam collapse rate in vitro and in vivo, and sensory assessment of the foam in the ear as follows:

### Foam Quality:

Visual inspection used to characterize the grade of the resulting foam was as follows:

Excellent (E)—very rich and creamy in appearance;

Good (G)—rich and creamy in appearance, but have bubble structure;

Poor (P)—not creamy, liquid in appearance.

### Agitability:

The test was used to determine whether the formulation moves freely within the canisters to allow the emulsion to be thoroughly mixed upon vigorous shaking. Canisters containing different formulations were shaken, and the sound obtained was used to grade the agitability of the formulation.

Good (G)—sound of vigorous shaking;

Poor (P)—sound of moderate shaking.

pH:

The pH was determined during the formulation preparation process and at the foam level. The pH was also measured at different time points during the accelerated stability studies.

### Centrifugation Test:

The test was aimed at assessing the emulsion stability at accelerated conditions. High speed centrifugation was used to mimic the phase separation process that would occur as a result of time. The test was performed as follows: About 5 gr of emulsion formulation was weighed in a scintillation vial and pentane was added up to a quantity equivalent to the quantity of propellant added for that foam formulation and vigorously mixed. The mixed formulation was left for more than 1 hr at room temperature, and then it was mixed and transferred into 1.5 ml microfuge tube. Air bubbles were removed from the tube and the tube was spun at 3,000 rpm for 10 min. The formulation appearance was graded as follows:

Creaming (Cr.)—Upper layer-opaque, Lower layer-transparent or translucent, reversible situation [% Creaming describes the percentage that occupies the upper layer portion].

Separation (Sep.)—More than two phases were observed and the situation was reversible upon shaking unless stated otherwise.

Homogeneous (Homog.)—No creaming, no separation.

### Foam Density:

Foam was released from the pressurized canister into a pre-weighed graduated cylinder (previously warmed to 37° C.) and foam was allowed to expand to its maximal volume. The weight and the volume of the foam were recorded. Foam density is defined as the weight recorded divided by the final volume after foam expansion. The test was performed and recorded 3 times independently. In case the results had more than +20% variability, additional 3 measurements were made and recorded. The average was taken as a final result.

### Foam Collapse Rate In Vitro:

Foam collapse rate in vitro was determined by delivering the foamable composition into a graduated 3 ml syringe that mimics the diameter and shape of the ear canal. The syringe was pre-warmed and maintained at 37° C. The final volume of the foam was recorded and considered as 100%. The syringe was further incubated at 37° C. for up to two hours. At different time points the remaining foam volume was assessed visually and expressed at percentage of the initial volume.

### Foam Collapse Rate In Vivo:

Foam was introduced in the ear canal of 4 healthy adult human subjects and the time of application was recorded. The percentage of the remaining foam was assessed at different time points for up to 3 hours. The subjects were allowed to continue their normal activities during the study.

### Sensory Assessment of the Foam in the Ear:

The human subjects that were submitted to the in vivo foam collapse rate study described above were requested to score various sensorial parameters related to the application and presence of the foam in the ear. The parameters were a) cold sensation when foam is applied; b) noise/“bubbling” as a result of foam expansion and initial collapse; c) hearing loss as a result of foam presence; d) general sensation of comfort and well-being when the foam is in the ear; and e) open remarks on foam sensorial feeling.

### HPLC to Determine the Ciprofloxacin Content in the Formulations:

To determine the amount of Ciprofloxacin present in the foam formulations, HPLC was performed for the pre-foam emulsions as well as for the foam formulations. Samples of 10 μl were applied to a YMC ODS AM 1207 column (5 μm, 4.0×100 mm) under a flow rate of 1 ml/min of 25 mM H₃PO₄ in Triethylamine, pH 3: acetonitrile (82:18), for 30 minutes run.

### Foam Development:

Table 2 lists the excipients used for formulation development.

TABLE 2
Excipients of formulation.

| | |
|---|---|
| Excipient | Role in formulation |

Oil phase

| | |
|---|---|
| Isopropyl myristate | Co-solvent, emollient |
| Mineral oil | Oil, emollient |
| Benzyl alcohol | Preservative |
| Cetyl alcohol | Emulsifier, thickener |
| Cetostearyl alcohol | Emulsifier, thickener |
| Glyceryl stearate | Consisting agent, surfactant, emulsifier |
| Lecithin | Emulsifier |
| Polyoxyl 40 stearate | Consisting agent, surfactant |
| Polysorbate 20 | Consisting agent, surfactant |
| Polysorbate 80 | Consisting agent, surfactant |

Water phase

| | |
|---|---|
| Hydroxyethyl cellulose | Polymer, stabilizing agent, |
| Polyvinyl alcohol (PVA) | Polymer, Stabilizing agent |
| Water | Solvent |
| Propylene glycol | Co-solvent, hydroscopic agent |
| Glycerin | Co-solvent, hydroscopic agent |
| Sodium acetate | pH modifying agent |
| Acetic acid | pH modifying agent |
| Ciprofloxacin HCl | Active Ingredient |

Foaming Agent

| | |
|---|---|
| Propane, Butane, Isobutane | Propellant |

### General Procedure for Formulation Preparation:

### Preparation of Water-Based Foam Formulations:

Step 1. Water phase preparation: A polymer (Hydroxyethylcellulose or Polyvinyl alcohol (PVA)) was dispersed in hot water (depending on the polymer used) with vigorous agitation until evenly dispersed and no clumps were detected. Other water phase excipients (as detailed in Table 2 herein above) were added according to the formulation composition and heated to 65-70° C. while mixing to obtain complete dissolution of all ingredients

Step 2. Oil phase preparation: Oil phase ingredients were heated to 70° C. until complete melting and mixed.

Step 3. Emulsification: The oil phase (step 2) was added slowly to the water phase (step 1) at 65-70° C. with vigorous mixing until uniform emulsion was obtained (the exact RPM used depended on batch size).

Step 4. Cooling and pH adjustment: The emulsion was cooled down and the temperature was kept at less than 30° C. The pH was then determined. If the pH was found to be higher than 5, acetic acid was added while mixing to obtain a pH of 4.5±0.5. The weight of the emulsion was determined. Water was added if necessary to obtain the correct weight.

Step 5. Filling and crimping of canisters. Each aerosol canister was filled with the emulsion slurry and crimped with valves using a manual crimper.

Step 6. Pressurizing: Pressurizing was carried out using a gas mixture composed of Propane: Butane: Isobutane (55:30:15). Canisters were filled with suitable amount of propellant (by weight).

### Preparation of Foam Formulations Devoid of Water:

Formulations devoid of water were prepared as follows: all water phase ingredients (not including Sodium Acetate, Acetic Acid and Ciprofloxacin HCl) were dissolved in Propylene Glycol (PG), not in water. The oil Phase was prepared as detailed herein above for the water containing emulsions: all oil phase ingredients were melted by stirring at 70° C. Emulsification was performed by slowly adding the oil phase to the PG phase at 70° C. with high mixing for more than 20 minutes. Weight correction was done with PG. Ciprofloxacin HCl was slowly added to the emulsion and mixed for 20 minutes. No pH adjustment was performed.

### Results

Seven formulations were initially prepared. Formulations #1 to #7 (designated herein below F01-F07) were oil-in-water emulsions. The oil phase in each formulation contained oil (6-10%) and non-ionic surfactants (5.5-8.5%). Water phase contained a polymer, a stabilizing agent, propylene glycol (PG) and glycerin as hygroscopic and viscosity increasing agents, sodium acetate and acetic acid as a buffering system, and Ciprofloxacin HCl. Formulation #12 (F12) is a formulation devoid of water in which PG served as the primary solvent to replace water. The formulations and their characteristics are presented in Tables 3 and 4, respectively.

TABLE 3
The constituents of formulations F01 to F11 (numbers represent percentage of
the ingredient in the formulation)

| | Formulation# | | | | | | | |
|---|---|---|---|---|---|---|---|---|
| Ingredient | F01 | F02 | F03 | F04 | F05 | F06 | F07 | F12 |
| Isopropyl myristate | 10 | 10 | 10 | | | | | |
| Mineral oil | | | | 6 | 6 | 6 | 6 | 5 |
| Benzyl alcohol | 0.3 | 0.3 | 0.3 | | | | | |
| Cetostearyl alcohol | 1 | 1 | 2 | 0.5 | 1 | 3 | 2 | 2 |
| Glyceryl stearate | | | 0.5 | 1 | 0.5 | 0.5 | 0.5 | 1 |
| Lecithin | | | | | | | | |
| Polyoxyl 40 stearate | | | | 3 | 3 | | 3 | 4 |
| Polysorbate 20 | 2 | 2 | | | | 4 | 1 | |
| Polysorbate 80 | 3 | 3 | 5 | 1 | 1 | | | |
| Hydroxyethyl cellulose | 0.25 | 0.25 | 0.25 | 1 | 0.5 | 0.25 | | 0.5 |
| PVA | 1 | 1 | 2 | | | 2 | 2 | |
| Propylene glycol | 15 | 15 | 15 | 3 | 4 | 10 | 5 | 79.2 |
| Glycerin | 15 | 15 | 15 | 8 | 8 | 10 | 10 | 8 |
| Sodium acetate | | 0.23 | 0.22 | 0.2 | 0.2 | 0.2 | 0.2 | |
| Acetic acid | | s.q. | s.q. | s.q. | s.q. | s.q. | s.q. | |
| Ciprofloxacin HCl | | 0.35 | 0.35 | 0.35 | 0.35 | 0.35 | 0.35 | 0.35 |
| Water | 52.4 | 51.9 | 49.4 | 76 | 75.5 | 63.75 | 70 | |

s.q.—Sufficient quantity

TABLE 4
Characteristics of foam formulations.

| | F01 | F02 | F03 | F04 | F05 | F06 | F07 | F12 |
|---|---|---|---|---|---|---|---|---|
| Propellant (%) | 8 | 8 | 8 | 8 | 8 | 8 | 8 | 8 |
| Foam quality | E | E | G | E | E | E | E | G |
| Agitability | G | G | G | G | G | G | G | G |
| pH | 4.95 | 4.60 | 4.60 | 4.56 | 4.43 | 4.48 | 4.49 | NA |
| Appearance | Cr. | Cr.-40% | Cr.-40% | Cr.-50% | Sep. | Sep. | Sep. | Cr.-60% |
| after | | | | | | | | |
| centrifugation | | | | | | | | |
| Density (g/ml) | ND | ND | 0.06 | 0.07 | 0.03 | 0.05 | 0.12 | ND |

NA—Not available, the test could not be performed.
ND—Not determined

Formulations F01 to F07 resulted in good or excellent foams having good agitability. The pH was within the required range in all formulations. After centrifugation formulations F05-F07 resulted in separation which was reversible upon shaking. All other formulations showed “creaming” to different degree and reversibility upon shaking. The density of the relatively stable foams tested was in the range of 0.02-0.07mg/ml.

To increase foam density additional formulations were prepared with different amounts of propellant. Tables 5 and 6 present the different formulations and their foam characteristics, respectively.

TABLE 5
Additional Formulations F13 to F17.

| | F13* | F14 | F15 | F16 | F17 |
|---|---|---|---|---|---|
| Oil-Phase | | | | | |
| Mineral oil | 6 | 6 | 6 | 5 | 6 |
| Cetyl alcohol | 1 | 1 | 1.5 | 1 | 1 |
| Glyceryl stearate | | | 0.5 | 0.5 | 0.25 |
| Lecithin | 2 | 2 | | | |
| Polysorbate 80 | 3 | 3 | 5 | 4 | 4 |
| Water-Phase | | | | | |
| Hydroxyethyl cellulose | 0.25 | 0.25 | 0.25 | 0.25 | 0.25 |
| PVA | 1 | 1 | 0.5 | 1 | |
| Propylene glycol | 8 | 8 | 10 | 10 | 80.2 |
| Glycerin | 8 | 8 | 8 | 8 | 8 |
| Sodium acetate | 0.2 | 0.2 | 0.2 | 0.2 | |
| Acetic acid | s.q. | s.q. | s.q. | s.q. | |
| Ciprofloxacin HCl | 0.35 | 0.35 | 0.35 | 0.3 | 0.35 |
| Water | 70.25 | 70.25 | 67.75 | 69.75 | |

*Nano-sized
s.q. Sufficient quantity

Formulation F13 had the same composition as F14, but its preparation procedure involved nano-sizing of the emulsion performed by a Gaulin high pressure homogenizer. Formulation F17 was a formulation devoid of water.

TABLE 6
Characterization of formulations F13 to F17.

| | F13 | F14 | | F15 | | | F16 | F17 |
|---|---|---|---|---|---|---|---|---|
| Propellant (%) | 8 | 4 | 6 | 2 | 4 | 6 | 2 | 6 |
| Foam quality | G | E | E | E | E | E | G | P |
| Agitability | G | G | G | G | G | G | G | G |
| pH | 5.03 | 5.03 | | 4.68 | | | 4.67 | NA |
| Appearance after | Sep. | Sep. | Sep. | Cr.- | Cr.- | Cr.- | Cr.- | Cr.- |
| centrifugation | | | | 60% | 65% | 60% | 30% | 40% |
| Density (g/ml) | ND | 0.09 | 0.07 | 0.17 | 0.15 | 0.07 | 0.27 | NA |

NA—Not available, the test could not be performed.
ND—Not determined

As indicated in Table 6, reducing propellant amount in the formulation did not affect significantly foam quality, but increased its density (formulations F14 and F15). The foam quality of F14 was better than F13 indicating that nano-sizing of the formulation does not improve its quality. Interestingly, the nano-sized formulation F13 had a better sensorial profile and different in vivo collapse rate than the counterpart formulation that did not undergo nano-sizing. The formulations devoid of water resulted in poor foam quality.

Formulations F4, F5, and F14 were selected for further optimization. As a result, formulations F18, F19, F22, and F21 were developed. The composition of each of the optimized formulation is summarized in Table 7.

TABLE 7
Composition of optimized formulations F18 to F23.

| | F18 | F19 | F21 | F22 | F23 |
|---|---|---|---|---|---|
| Oil Phase | | | | | |
| MCT oil | | | | | 15 |
| Mineral oil | 8 | 6 | 6 | 10 | |
| Cetyl alcohol | 1 | 0.5 | 1 | 1 | |
| Glyceryl stearate | 0.5 | 1 | 0.5 | 0.5 | |
| Lecithin | | | 2 | | 2.6 |
| Polyoxyl 40 stearate | | 3 | | 2.6 | |
| Polysorbate 80 | 4 | 1 | 3 | 0.8 | 2.5 |
| Water-Phase | | | | | |
| Hydroxyethyl cellulose | 0.3 | 1 | 0.3 | 0.5 | 1 |
| PVA | 1 | | 1.5 | | |
| Propylene glycol | 6 | 3 | 6 | 3 | 3 |
| Glycerin | 6 | 8 | 6 | 3 | 3 |
| Sodium acetate | 0.2 | 0.2 | 0.2 | 0.2 | 0.2 |
| Acetic acid | s.q. | s.q. | s.q. | s.q. | s.q. |
| Ciprofloxacin HCl | 0.35 | 0.35 | 0.35 | 0.35 | 0.35 |
| Water | 73 | 76 | 74 | 78 | 73 |

s.q. Sufficient quantity

Formulation F23 tested the use of MCT instead of mineral oil. Each formulation was prepared with two propellant concentrations. The characterization of the formulations obtained is presented in table 8.

TABLE 8
Characterization of optimized formulations.

| | F18 | | F19 | | F21 | | F22 | | F23 | |
|---|---|---|---|---|---|---|---|---|---|---|
| Propellant (%) | 2 | 4 | 2 | 4 | 2 | 4 | 4 | 6 | 4 | 6 |
| Foam quality | P | E | G | E | P | G | G | E | G | E |
| Agitability | G | G | G | G | G | G | G | G | G | P |
| pH | 4.7 | | 4.5 | | 4.3 | | 4.7 | | 4.93 | |
| Appearance after | ND | Sep. | ND | Sep. | ND | Sep. | Homog | ND | Cr. | ND |
| centrifugation | | | | | | | | | | |
| Density (g/ml) | NA | 0.12 | 0.30 | 0.15 | NA | 0.12 | 0.15 | 0.12 | 0.16 | 0.10 |

NA—Not available;
ND—Not determined.

After centrifugation, formulations F18, F19 and F21 showed separation which was reversible upon shaking. Formulation F23 showed creaming while F22 remained homogenous after centrifugation. Formulations with 2% propellant resulted in poor foams.

### Physical Measurements and Sensorial Evaluation:

The physical characteristics and sensorial evaluation are presented in Table 9. Based on the physical foam parameters as presented in tables 4, 6, and 8 herein above, several foam formulations were selected for a collapse rate testing in ears of healthy human subjects.

TABLE 9
Collapse rate testing and sensory evaluation.

| | | | | | Overall | Overall Foam |
|---|---|---|---|---|---|---|
| | | | | | Sensorial | Score for Otic |
| | | | | Noise when | Feeling | use [+ = poor, |
| Formulation | Propellant | Collapse % | Hearing | foam is | [1 = poor - | ++ = fair, |
| # | [%] | after 3 hours | Quality | applied | 5 = excellent] | +++ = excellent] |
| F14 | 4% | 60% collapse | Good | Minimal | 4 | +++ |
| F15 | 2% | 0% | Reduced | Minimal | 1 | + |
| | | (no collapse) | hearing | | | |
| F15 | 4% | 0% | Reduced | Minimal | 1 | + |
| | | (no collapse) | hearing | | | |
| F16 | 4% | 10% collapse | Reduced | Minimal | 2 | + |
| | | | hearing | | | |
| F18 | 4% | 20% collapse | Reduced | Minimal | 3 | ++ |
| | | at ear canal | hearing | | | |
| | | entrance | | | | |
| | | 50% collapse | | | | |
| | | inside the ear | | | | |
| | | canal | | | | |
| F19 | 4% | 80% collapse | Reduced | Minimal | 2 | ++ |
| | | | hearing | | | |
| F21 | 4% | 50% collapse | Good | Minimal | 5 | +++ |
| | | at ear canal | | | | |
| | | entrance | | | | |
| | | % collapse 90 | | | | |
| | | inside the ear | | | | |
| | | canal | | | | |
| F22 | 4% | 20% collapse | Good | Minimal | 3 | ++ |
| F22 | 8% | 50% collapse | Good | Minimal | 3 | ++ |
| | | at ear canal | | | | |
| | | entrance | | | | |
| | | % collapse 90 | | | | |
| | | inside the ear | | | | |
| | | canal | | | | |

Strikingly, there was a very low correlation in the collapse rate between the in vitro and the in vivo measurements. In the in vivo studies foam formulations collapsed at a much faster rate than in the in vitro studies despite the fact that both were measured at the same temperature (37° C.). Without wishing to be bound to any mechanism of action, it is postulated that the faster foam collapse in vivo was due to the fact that humans move their jaws while speaking, sipping, chewing, etc. The jaw movement causes the walls of the ear canal to contract and expand slightly leading to a mechanical pressure on the foam that increased its collapse rate. The sensorial feeling upon application of the foam in the ear in human subjects was then evaluated. As seen in Table 9 herein above, none of the human subjects reported a “cold feeling” or dizziness when the foam was applied in the ear, a clear advantage over ear drops that may cause dizziness when applied into the ear.

Selection criteria for the most suitable foam formulations were as follows: high foam density that does not cause reduced hearing; significant collapsing after 3 hours; and general comfortable sensation when the foam is applied in the ear. Among the foams tested, Formulation F15, F16, F18, and F22 showed delayed collapse in the ear implicating that these foam formulations are slightly less advantageous for pharmaceutical topical applications. The formulations F14 with 4% propellant, F21 with 4% propellant and F22 with 8% propellant, all showed the selection criteria and therefore tested for accelerated stability and compatibility with the packaging materials.

### Preparation of Selected Foam Formulations:

### Formulation #14

RPM rate and propeller size (50 mm) specified herein below were used for the preparation of the formulations of a batch size equal to about 100 g.

Step 1. Water phase preparation.
1.1 Polyvinylalcohol was dissolved in water under vigorous agitation at 500-550 rpm until clear solution was obtained and no clumps were detected.
1.2 Hydroxyethylcellulose was added slowly while mixing vigorously and heating the water to 80° C. maintaining the mixing conditions similar to those in 1.1 until thoroughly wetted and evenly dispersed and no clumps were observed.
1.3 Propylene glycol and glycerin were added and mixed for 10 minutes while maintaining the temperature at 60° C.
   Step 2. Oil phase preparation.
2.1 Mineral oil and Cetyl alcohol were heated to 70-80° C. until complete melting and clear solution was obtained.
2.2 The solution was cooled to 50° C. and Polysorbate 80 was added.
2.3 Lecithin was added and mixed with high-shear homogenizer at 7500-8000 rpm until homogeneity was obtained.

### Step 3. Emulsification.

3.1 The oil phase (step 2) was added to the water phase (step 1) at 50-55° C. with vigorous agitation at 550-600 rpm.
3.2 Agitation continued for at least 10 minutes until uniform emulsion was obtained as determined by visual inspection.
   Step 4. Cooling, active pharmaceutical ingredient addition and pH adjustment.
4.1 The emulsion was cooled to 25-30° C. while mixing at 550-600 rpm.
4.2 Sodium acetate and Acetic acid were added while mixing to adjust the pH to 4.75±0.25.
4.3 Ciprofloxacin HCl was added while mixing for at least 10 minutes.
4.4 The final weight of the emulsion was measured and water was added if required to obtain the correct weight.
4.5 The pH was adjusted to 4.75±0.25, if required.

### Formulation #21

RPM rate specified in the preparation procedure refers to propeller size of 50 mm and batch size of 100 g.

Step 1. Water phase preparation.
1.1 Polyvinylalcohol was dissolved in water under vigorous agitation at 500-550rpm until clear solution and no clumps were detected.
1.2 Hydroxyethylcellulose was added slowly while mixing with vigorous agitation (as in the previous step) until evenly dispersed and no clumps were observed.
1.3 Propylene glycol and Glycerin were added and mixed for 10 minutes. The temperature was maintained at 60° C.
   Step 2. Oil phase preparation.
2.1 Mineral oil, Glyceryl stearate and Cetyl alcohol were heated to 70-80° C. until complete melting and clear solution was obtained.
2.2 The solution was cooled to 50° C. and Polysorbate 80 was added.
2.3 Lecithin was added and mixed in a high-shear homogenizer at 7500-8000rpm until homogeneity was obtained.

### Step 3. Emulsification.

3.1 The oil phase (step 2) was added slowly to the water phase (step 1) at 50-55° C. with vigorously agitation at 550-600rpm.
3.2 Agitation continued for at least 10 minutes until uniform emulsion was obtained as determined by visual inspection.
   Step 4. Cooling, active pharmaceutical ingredient addition and pH adjustment.
4.1 The emulsion was cooled to 25-30° C. while mixing at 550-600rpm.
4.2 Sodium acetate and Acetic acid were added while mixing to adjust pH to 4.75±0.25.
4.3 Ciprofloxacin HCl was added while mixing which continued for at least 10 minutes.
4.4 The final weight of the emulsion was determined and water was added if required to obtain the correct weight.
4.5 The pH was adjusted to 4.75±0.25, if required.

### Formulation #22

RPM rate specified in the preparation procedure refers to propeller size of 50 mm and batch size of 100 g.

Step 1. Water phase preparation.
1.1 Hydroxyethylcellulose was added to water under vigorous agitation at 500-550rpm until evenly dispersed and no clumps were detected. Water was then heated to 70° C.
1.2 Propylene glycol and Glycerin were added. Mixing was continued for 10 minutes and the temperature was maintained at 60-65° C.
   Step 2. Oil phase preparation.
2.1 Mineral oil, Glyceryl monostearate, Polyoxyl 40 stearate and Cetyl alcohol were heated together to 70-80° C. until complete melting and clear solution was obtained.
2.2 The solution was cooled to 65° C. and Polysorbate 80 was added.

### Step 3. Emulsification.

3.1 The oil phase (step 2) was added slowly to the water phase (step 1) at 60-65° C. with vigorous agitation at 600-650rpm.
3.2 Agitation continued for at least 10 minutes until uniform emulsion was obtained as determined by visual inspection.
   Step 4. Cooling, active pharmaceutical ingredient addition and pH adjustment.
4.1 The emulsion was cooled to 25-30° C. while mixing at 600-650rpm.
4.2 Sodium acetate and Acetic acid were added while mixing to adjust pH to 4.75±0.25.
4.3 Ciprofloxacin HCl was added while mixing which continued for at least 10 min.
4.4 The final weight of the emulsion was measured and water was added if required to obtain the correct weight.
4.5 The pH wad adjusted to 4.75±0.25, if required.

### EXAMPLE 3

### Formulation Stability Studies at Accelerated Aging Conditions

Stability studies at accelerated aging conditions were performed on formulations #14, #21 and #22. The formulations were packed in aluminum canisters coated with various varnishes, crimped with commercial valves and incubated at 25° C., 40° C. and at 50° C. for different periods of time. Canisters were stored for 1 month at 50° C. (indicative of 6 months stability at room temperature) and for 3 months at 40° C. (indicative of 9-12 months stability at room temperature) as well as for 3, 9, and 14 months at 25° C. Samples from the formulations were taken at zero time (0 months) and at the indicated time points and tested for their physical and chemical properties such as: ciprofloxacin content (using the HPLC-assay described herein above), pH, foam quality, foam density and collapse rate. Tables 10-12 summarize the results of Ciprofloxacin content in the formulations tested upon incubation at accelerated aging conditions.

TABLE 10
Ciprofloxacin content at 25° C.

| | Form #14 | Form #21 | Form #22 |
|---|---|---|---|
| Time | % Ciprofloxacin | % Ciprofloxacin | % Ciprofloxacin |
| [months] | in formulation | in formulation | in formulation |
| 0 | 0.32 | 0.31 | 0.3 |
| 1 | 0.31 | 0.31 | 0.32 |
| 3 | 0.31 | 0.3 | 0.29 |

TABLE 11
Ciprofloxacin content at 40° C.

| | Form #14 | Form #21 | Form #22 |
|---|---|---|---|
| Time | % Ciprofloxacin | % Ciprofloxacin | % Ciprofloxacin |
| [months] | in formulation | in formulation | in formulation |
| 0 | 0.32 | 0.31 | 0.3 |
| 1 | 0.31 | 0.31 | 0.31 |
| 3 | 0.29 | 0.28 | 0.29 |

TABLE 12
Ciprofloxacin content at 50° C.
Incubation Conditions 50° C.

| | Form #14 | Form #21 | Form #22 |
|---|---|---|---|
| Time | % Ciprofloxacin | % Ciprofloxacin | % Ciprofloxacin |
| [months] | in formulation | in formulation | in formulation |
| 0 | 0.32 | 0.31 | 0.3 |
| 1 | 0.31 | 0.3 | 0.31 |

The results from Tables 10-12 show that the amount of Ciprofloxacin did not significantly change upon incubation at 40° C. or 50° C. for 1 or 3 months. The occasional variations (+/−15%) found were due to the sample extraction method and not to actual decreases in ciprofloxacin content.

Likewise, no significant changes were found in the pH, foam quality, foam density, collapse rate, and microbiological activity of formulation #21 when incubated for 9 or 14 months at 25° C.

These results thus demonstrate that the formulations developed are stable and display a long shelf-life.

### EXAMPLE 4

### Compatibility Studies Between Otic Formulations and Packaging

Compatibility studies between the formulations (#14, #21 and #22) and the packaging materials, i.e., aluminum canister and valves, were performed. Upon incubation of the formulations with the packaging materials for 1 month at 50° C. and 3 months at 40° C., no noticeable changes were found in the coating of the aluminum canisters as well as in the coating and inner parts that compose the crimped valves.

### EXAMPLE 5

### Assessment of the Safety of the Formulations in Animals and Humans

The otic formulations #14 #21 and #22 were then tested for dermal irritation and delayed contact hypersensitivity potential in albino rabbits in compliance with the OECD Good Laboratory Practice (OECD Environmental Health and Safety Publications; Series on Principles of Good Laboratory Practice and Compliance Monitoring—Number 1 ENV/MC/CHEM (98) 17, as revised in 1997; and according to the International Standard ISO 10993-10:2002 (E) Second Edition, 1 Sep. 2002, Biological Evaluation of Medical Devices-Part 10.

Single occlusive skin applications took place for 4 hours to 6 male NZW rabbits (3 rabbits were exposed to the formulations #14 while the other 3 rabbits were exposed to formulations #21 and #22). At the end of the exposure period, dressings with the residual formulations were removed using lukewarm tap water. Dermal reactions were scored and recorded at 1, 24, 48 and 72 hours after patch removal. No skin reactions (edema or erythema) were noted in any of the rabbits at the evaluation points tested. No clinical signs in reaction to the treatments were evident or no abnormal changes in body weight were noted in the rabbits throughout the entire study period. The results indicated that Formulations #14, #21 and #22 are safe and have a Primary Irritation Index (PII) of 0.0 (the lowest score possible), i.e., classifying the irritation potential of the formulations as “negligible” to the rabbit's skin.

Additional safety studies were performed in humans using the Human Repeated Insult Patch Test also known as HRIPT (Draize et al., J. Pharm. Exp. Ther. 83: 377-390, 1944). The test challenges the development of Allergic Contact Dermatitis upon repeated applications of a test item under exaggerated exposure conditions.

In a study performed in compliance with Good Clinical Practice standards, formulations F14 and F21 were applied on the intrascapular region of the back or on the arm of fifty human volunteers (16 males and 34 females) aged 18 to 65 years using the HRIPT. The human volunteers were determined to be in good general health and free of any visible skin disease or anomaly in the area to be patched.

During the Induction Phase, the patch was applied to a designated contact site and remained in place for 24 hours. At the end of this period, the patch was removed and the site was examined for any dermal response. The subject rested for 24 hours, after which the skin site was examined again. A patch was then applied to the same site as previously used. The second application was identical to the first and remained in place for 24 hours. The procedure was repeated twice a week until a series of nine applications were made. The patch site was examined for any dermal response. The same site was used throughout the study.

During the Challenge Phase, after the 9th application, a rest period of 2 weeks elapsed after which a challenge application was applied in the same manner and to the same site described herein above and to a naïve site on the opposite site of the body. The challenge application was removed after 24 hours and the site was examined and graded for signs of irritation or sensitization. A follow-up examination was conducted at 24 hours after patch removal as well as at 48 hours and 72 hours.

The dermal responses were scored according to the following grading scale:

Erythema Scale:
0 No visible erythema
1 Mild eryhtema (faint pink to definite pink)
2 Moderate erythema (definite redness)
3 Severe erythema (very intense redness)

Designations for elevated responses: Edema, papules, vesicles, and bullae, if present, are graded as independent responses.
E—Edema—definite swelling
P—Papules—many small, red, solid elevations—surface of reaction has granular feeling.
V—vesicles—small, circumscribed elevations having translucent surfaces so that fluid is visible (blister like).
B—Bullae—vesicles with a diameter>0.5 cm. Vesicles may coalesce to form one or a few large blisters that fill the patch site.
NT Not tested.

The dermal responses of the 50 volunteers in all the applications and on all the sites were graded as 0 (no visible erythema or elevated response). None of the volunteers developed any irritation or skin sensitization sign during the induction or challenge phases. The HRIPT results clearly indicated that the formulations do not sensitize the skin or produce allergic responses upon repeated applications to the same site. Taken together, the results demonstrated that the formulations are safe for human use. Moreover, as treatments with otic foams are expected to last a few days and may require repeated application of the medication on the same site (the inflamed ear), these results indicate that the formulations can be used for long periods of time without causing any irritation or any allergic responses.

### EXAMPLE 6

### In Vitro Antibiotic Efficacy Studies

In order to demonstrate that the excipients used in the formulations (#14, #21 and #22) do not interact with Ciprofloxacin or inhibit its antibiotic activity, an Antibiotics-Microbial Assay—Zone of Growth Inhibition was performed. In this assay the diameter (halo) of bacterial growth inhibition around filters soaked with a test item is measured and compared to a standard solution of antibiotic. For this end, Escherichia coli was grown overnight on liquid LB broth (5 g/l yeast extract, 10 g/l tryptone/peptone, 10 g/l NaCl). Sterile Microbiological cotton swabs were soaked in the bacterial cultures and evenly spread on top of LB-agar plates. Filters soaked with 5 μl or 10 μl of the tested formulations were placed on top of the plates and the plates were further incubated for 24 hrs. The diameter (halo) of growth inhibition around each filter was measured after 24 hrs of incubation at 37° C. Ciloxan® eyes and ear drops (Alcon Laboratories) that contain the same concentration of Ciprofloxacin (0.3%) as in the formulations tested was used as a control.

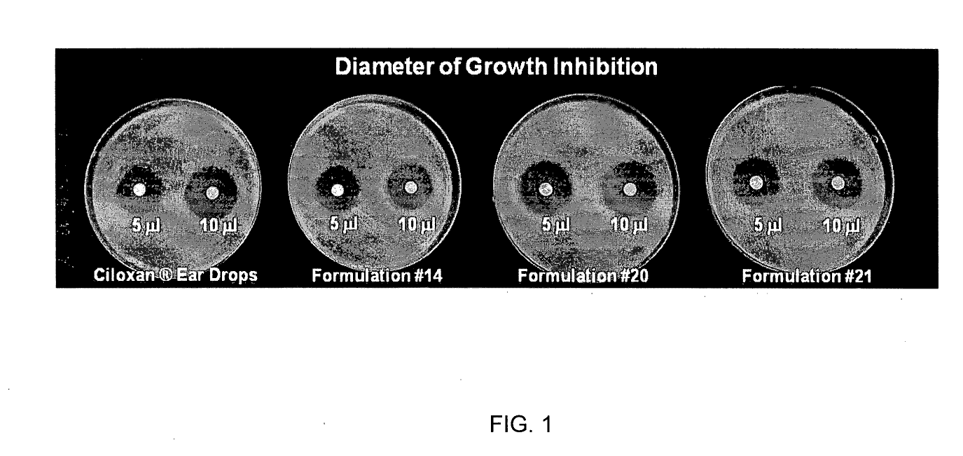

The results are summarized in Table 13 and in FIG. 1.

TABLE 13
Effect of different formulations on the diameter of growth inhibition.

| | 5 microliter of test item | | 10 microliter of test item | |
|---|---|---|---|---|
| | Diameter | Inhibition | Diameter | Inhibition |
| | of growth | diameter | of growth | diameter |
| | inhibition | compared to | inhibition | compared to |
| Formulation | [cm] | control [%] | [cm] | control [%] |
| CILOXAN | 2.4 | 100.00 | 2.7 | 100.0 |
| (control) | | | | |
| Formulation #14 | 2.3 | 95.83 | 2.75 | 101.9 |
| Formulation #21 | 2.45 | 102.08 | 2.55 | 94.4 |
| Formulation #22 | 2.3 | 95.83 | 2.5 | 92.6 |

The results demonstrate that the antibiotic potency of the formulations tested is similar to the potency displayed by the standard Ciloxan® solution (Table 13 and FIG. 1).

### EXAMPLE 7

### Clinical Trial for Formulation #21

A clinical study for assessing safety, efficacy, and clinical equivalence of formulation #21 in comparison to commercial ciprofloxacin ear drops (Ciloxan of Alcon Labs) in the treatment of acute diffuse Otitis Externa (“Swimmer's ear”) was conducted. The study, open-label and randomized, enrolled 63 adult patients diagnosed with Otitis Externa of presumed bacterial origin. Inclusion and outcome criteria included the signs and symptoms of the disease: ear edema, ear pain, ear discharge, and tenderness to movement of the tragus/pinna. The study, complying with GCP guideline, was performed in four medical centers in Israel from May to October 2009. The patients were randomized into one of the two treatment groups: (1) Foam formulation #21 (n=32) or (2) Commercial ear drops (Ciloxan of Alcon Labs; n=31). Each medication contained ciprofloxacin at the same concentration.

The patients applied the medications twice a day for 7 days. Upon completion of the treatment, the patients returned for a test-of-cure visit in which the status of the disease was assessed and compared to baseline. Concomitant antibiotic treatments were not allowed during the study. The patients were considered Cured if all signs and symptoms of the disease were fully resolved (Resolution) or if not all the symptoms were fully resolved but no additional antibiotics treatment was needed (Improvement). The patients were considered Treatment Failure if additional antibiotic therapy was needed.

The results in Table 14 showed that the percentage of cured patients was 93.8% in the foam treated group and 93.6% in the Ciloxan Group. Treatment failure was 6.2% in the foam treated group and 6.4% in the Ciloxan group and the cure/failure ratio was not different (p=0.999). The per-protocol analysis (PP) included 57 patients (Foam formulation #21 n=29 and Ciloxan n=28) and showed 100% cure in both groups.

Moreover, the foam formulation was found to be more effective than Ciloxan as reflected in the complete resolution of signs and symptoms of the disease (see Table 14, 81.3% vs. 71% respectively), in the resolution of otic discharge (see Table 14, 100% vs.

84.6%, respectively) and in the reduction of at least 50% in ear pain after 3-4 days of treatment (73.3% vs. 58.3%, respectively). These results suggest that the onset of pain reduction is faster when treated with the foam formulation compared to the Ciloxan ear drops.

TABLE 14
Summary of the clinical results

| | | Ciprofloxacin |
|---|---|---|
| Clinical Response | Formulation #21 | Ear Drops |
| | (n = 32) | (n = 31) |
| Cured (Resolution + | 93.8% * | 93.6% * |
| Improvement) | | |
| Failure | 6.2% | 6.4% |
| Resolution | 81.3% | 71% |
| | (n = 29) | (n = 28) |
| Resolution of Otic | 100% | 84.6% |
| Discharge | | |
| Percentage of Pain | 73.3% | 58.3% |
| responders at Visit 2 (**) | | |

* p = 0.999
** Reduction in VAS score of at least 50% from baseline
(Per protocol Analysis, n = 57)

It will be appreciated by persons skilled in the art that the present invention is not limited by what has been particularly shown and described herein above. Rather the scope of the invention is defined by the claims that follow.

1-29. (canceled)

30. A foamable otic pharmaceutical composition comprising:
(a) an oil-in-water emulsion comprising:
(i) a fluoroquinolone in an amount effective for antibacterial action;
(ii) a hydrophobic solvent;
(iii) an emulsifier and/or a synthetic surfactant;
(iv) a stabilizing agent;
(v) a polar co-solvent in an amount ranging from about of 5% to about 30% (w/w);
(vi) water in an amount ranging from about 50% to about 80% (w/w); and
(b) a compressed propellant gas;
wherein the composition packaged in a container is adapted to form foam after dispensing from the container, the foam having a density of about 0.1 gr/ml to about 0.5 gr/ml.

31. The foamable otic pharmaceutical composition according to claim 30, wherein the fluoroquinolone is selected from the group consisting ciprofloxacin, ofloxacin, moxifloxacin, levofloxacin, marbofloxacin, enrofloxacin, and salts thereof.

32. The foamable otic pharmaceutical composition according to claim 30, wherein the oil-in-water emulsion comprises:
(i) ciprofloxacin in an amount ranging from about 0.1% to about 0.5% (w/w);
(ii) a hydrophobic solvent;
(iii) an emulsifier;
(iv) a synthetic surfactant;
(v) a stabilizing agent;
(vi) a polar co-solvent in an amount ranging from about 5% to about 30% (w/w);
(vii) a buffering system; and
(viii) water in an amount ranging from about 50% to about 80% (w/w).

33. The foamable otic pharmaceutical composition according to claim 32, wherein the oil-in-water emulsion comprises:
(i) ciprofloxacin hydrochloride in an amount ranging from about 0.2% to about 0.35% (w/w);
(ii) a hydrophobic solvent in an amount ranging from about 5% to about 15% (w/w);
(iii) an emulsifier in an amount ranging from about 0.1% to about 10% (w/w);
(iv) a synthetic surfactant in an amount ranging from about 0.1% to about 10% (w/w);
(v) a stabilizing agent in an amount ranging from about 0.1% to about 10% (w/w);
(vi) a polar co-solvent in amount ranging from about 5% to about 30%;
(vii) a buffering system; and
(viii) water in an amount ranging from about 50% to about 80%.

34. The foamable otic pharmaceutical composition according to claim 30, wherein the hydrophobic solvent is mineral oil present in the composition in an amount ranging from about 5% to about 15%.

35. The foamable otic pharmaceutical composition according to claim 30, wherein the emulsifier is selected from the group consisting of lecithin or a derivative thereof, phospholipids, long chain alcohols and combinations thereof, said emulsifier is present in the composition in an amount ranging from about 0.1% to about 10%.

36. The foamable otic pharmaceutical composition according to claim 30, wherein the surfactant is selected from the group consisting of polysorbate 20, polysorbate 60, polysorbate 80, glyceryl stearate, polyoxyl 40 stearate, and combinations thereof, said surfactant is present in the composition in an amount ranging from about 0.1% to about 10% (w/w).

37. The foamable otic pharmaceutical composition according to claim 30, wherein the stabilizing agent is selected from the group consisting of hydroxyethyl cellulose, polyvinyl alcohol, and combinations thereof, said stabilizing agent is present in the composition in an amount ranging from about 0.1% to about 10% (w/w).

38. The foamable otic pharmaceutical composition according to claim 30, wherein the polar co-solvent is selected from the group consisting of propylene glycol, glycerin, and combinations thereof, said polar co-solvent is present in the composition in an amount ranging from about 5% to about 30% (w/w).

39. The foamable otic pharmaceutical composition according to claim 30, wherein the buffering system comprises acetate buffer in an amount ranging from about 0.1% to about 1% (w/w).

40. The foamable otic pharmaceutical composition according to claim 30, wherein water is present in the composition from about 70% to about 80% (w/w).

41. The foamable otic pharmaceutical composition according to claim 30, wherein the pH of the composition ranges from about 4 to about 7.

42. The foamable otic pharmaceutical composition according to claim 30, wherein the compressed propellant gas is a mixture of hydrocarbons present in an amount ranging from about 1% to 8% by weight of the composition.

43. The foamable otic pharmaceutical composition according to claim 30, further comprising an anti-inflammatory steroid.

44. The foamable otic pharmaceutical composition according to claim 30, further comprising an antifungal agent.

45. The foamable otic pharmaceutical composition according to claim 30 comprising: ciprofloxacin HCl in an amount of 0.35% (w/w), mineral oil in an amount of 6% (w/w), cetyl alcohol in an amount of 1% (w/w), lecithin in an amount of 2% (w/w), polysorbate 80 in an amount of 3% (w/w), hydroxyethyl cellulose in an amount of 0.25% (w/w), polyvinyl alcohol in an amount of 1% (w/w), propylene glycol in an amount of 8% (w/w), glycerin in an amount of 8% (w/w), sodium acetate in an amount of 0.2% (w/w), acetic acid, and water in an amount of about 70% (w/w), wherein the pH of the pharmaceutical composition is of about 4.5 to about 5 before adding the compressed propellant gas in an amount of 4% to 6% by weight of the composition.

46. The foamable otic pharmaceutical composition according to claim 30 comprising: ciprofloxacin HCl in an amount of 0.35% (w/w), mineral oil in an amount of 6% (w/w), cetyl alcohol in an amount of 1% (w/w), glyceryl stearate in an amount of 0.5% (w/w), lecithin in an amount of 2% (w/w), polysorbate 80 in an amount of 3% (w/w), hydroxyethyl cellulose in an amount of 0.3%, polyvinyl alcohol in an amount of 1.5% (w/w), propylene glycol in an amount of 6% (w/w), glycerin in an amount of 6% (w/w), sodium acetate in an amount of 0.2% (w/w), acetic acid, and water in an amount of 74% (w/w), wherein the pH of the pharmaceutical composition is of about 4.5 to about 5 before adding the compressed propellant gas in an amount of 2% to about 4% by weight of the composition.

47. The foamable otic pharmaceutical composition according to claim 30 comprising: ciprofloxacin HCl in an amount of 0.35% (w/w), mineral oil in an amount of 10% (w/w), cetyl alcohol in an amount of 1% (w/w), glyceryl stearate in an amount of 0.5% (w/w), polyoxyl 40 stearate in an amount of 2.6% (w/w), polysorbate 80 in an amount of 0.8% (w/w), hydroxyethyl cellulose in an amount of 0.5% (w/w), propylene glycol in an amount of 3% (w/w), glycerin in an amount of 3% (w/w), sodium acetate in an amount of 0.2% (w/w), acetic acid, and water in an amount of 78% (w/w), wherein the pH of the pharmaceutical composition is of about 4.5 to about 5 before adding the compressed propellant gas in an amount of 4% to about 6% by weight of the composition.

48. A method for preparing a foamable otic pharmaceutical composition comprising: admixing a fluoroquinolone with an oil-in-water emulsion, and admixing a compressed propellant gas with the oil-in-water emulsion in a container accommodating a pressurized product, the propellant gas and said oil-in-water emulsion adapted to form foam after dispensing from the container, the foam having a density of about 0.1 g/ml to about 0.5 g/ml.

49. The method according to claim 48 comprising the following steps:
(a) preparing an aqueous solution comprising water, a stabilizing agent, and optionally a polar co-solvent;
(b) preparing an oil phase solution comprising a hydrophobic solvent, an emulsifier, and optionally a polar co-solvent and/or a synthetic surfactant;
(c) combining the oil phase solution of step (b) with the aqueous solution of step (a) to obtain an emulsion;
(d) admixing a fluoroquinolone with the emulsion of step (c); and
(e) admixing a compressed propellant gas with the emulsion in a container, wherein the propellant gas and the emulsion of step (d) adapted to form foam after dispensing from the container, the foam having a density of about 0.1 gr/ml to about 0.5 gr/ml.

50. A method for treating an ear disorder comprising administering to the ear of a subject in need of such treatment a therapeutically effective amount of a foamable otic pharmaceutical composition according to claim 30.
